(19) United States
(12) Patent Application Publication
(10) Pub. No.: US 2005/0287539 A1
(43) Pub. Date: Dec. 29, 2005

(54) Methods and compositions for preparing capped RNA

(76) Inventors: Emmanuel Labourier, Austin, TX (US); Brittan L. Pasloske, Austin, TX (US)

Correspondence Address: FULBRIGHT & JAWORSKI L.L.P., 600 CONGRESS AVE., SUITE 2400, AUSTIN, TX, 78701, US

(21) Appl. No.: 10/880,350

(22) Filed: Jun. 29, 2004

(51) Int. Cl. C12Q 1/68; C12P 19/34

(52) U.S. Cl. 435/6; 435/91.2

(57) ABSTRACT

The present invention concerns methods and compositions for increasing the yield of capped and full-length RNA transcripts produced in in vitro transcription reactions. Such methods and compositions can be used for cost-efficient, large scale production of capped full-length RNA transcripts that can be subsequently translated. Methods and compositions involve reaction conditions that promote such production, and includes the implementation of fed-batch introduction of GTP, which competes with a cap analog.

### BACKGROUND OF THE INVENTION

1. Field of the Invention

The present invention relates generally to the field of molecular biology. More particularly, it concerns methods and compositions for generating high yields of RNA transcripts that have a non-extending nucleotide at their 5′ end, such as a cap analog.

2. Description of Related Art

In vitro transcription, originally developed by Krieg and Melton (1987) for the synthesis of RNA using an RNA phage polymerase, is an integral part of the variety of techniques used in molecular biology. Typically these reactions include at least a phage RNA polymerase (T7, T3 or SP6), a DNA template containing a phage polymerase promoter, nucleotides (ATP, CTP, GTP and UTP), and a buffer containing a magnesium salt. Since an increase in the yield of these reactions would be beneficial in both time and expense, several groups worked to optimize the yields of RNA synthesized by in vitro transcription by increasing nucleotide concentrations, adjusting magnesium concentrations and by including inorganic pyrophosphatase (U.S. Pat. No. 5,256,555; Gurevich, 1991; Sampson, 1988; Wyatt, 1991). Such improvements have been incorporated into commercial kits for the large-scale synthesis of in vitro transcripts (MEGAscript®, Ambion, Inc.). The RNA synthesized in these reactions is usually characterized by a 5′ terminal nucleotide that has a triphosphate at the 5′ position of the ribose. Typically, depending on the RNA polymerase and promoter combination used, this nucleotide is a guanosine, although it can be an adenosine (see e.g., Coleman et al., 2004). In these reactions, all four nucleotides are typically included at equimolar concentrations and none of them is limiting.

The reactions described above are batch reactions—that is, all components are combined and then incubated at ˜37° C. to promote the polymerization of the RNA until the reaction terminates. Typically, most researchers use a batch reaction because of convenience and they obtain as much RNA as needed from such reactions for their experiments. However, there are applications where much greater quantities of RNA are required and therefore, efforts were undertaken by Kern (1997; 1999) to increase RNA yields at a reduced cost. These researchers developed a “fed-batch” system to increase the efficiency of the in vitro transcription reaction. All components were combined, but then additional amounts of some of the reagents were added over time, such as the nucleotides and magnesium, to try to maintain constant reaction conditions. In addition, the pH of the reaction was held at 7.4 by monitoring it over time and adding KOH as needed. The fed-batch strategy yielded a 100% improvement in RNA per unit of RNA polymerase or DNA template for a very short, 38 base-pair template. These researchers studied only the single reaction and did not consider what would happen in the context of more than one reaction. Furthermore, this method can be applied for synthesizing only in vitro transcripts containing a triphosphate at the 5′ terminus.

In eukaryotic cells, messenger RNA (mRNA) is the RNA directly translated by ribosomes to produce the encoded protein. mRNA carry a 5′ cap or N-7 methyl GpppG. The cap stabilizes the mRNA, protecting it from 5′ to 3′ exonuclease degradation and it enhances translation by promoting the interaction of the ribosome with the mRNA.

To synthesize a capped RNA by in vitro transcription, a cap analog (e.g., N-7 methyl GpppG or m7GpppG) is included in the transcription reaction. The RNA polymerase will incorporate the cap analog as readily as any of the other nucleotides, that is, there is no bias for the cap analog. However, the cap analog will be incorporated only at the 5′ terminus because it does not have a 5′ triphosphate. In the case of T7, T3 and SP6 RNA polymerase, the +1 nucleotide of their respective promoters is usually a G residue and if both GTP and m7GpppG are present in equal concentrations in the transcription reaction, then they each have an equal chance of being incorporated at the +1 position. Typically, 7mGpppG is present in these reactions at several-fold higher concentrations than the GTP to increase the chances that a transcript will have a 5′ cap. In Ambion's mMESSAGEmMACHINE® kit (Cat. #1344, Ambion, Inc.), it is recommended that the cap to GTP ratio be 4:1 (6 mM: 1.5 mM). Using these conditions, the transcription reaction will yield ˜80% capped RNA and 20% uncapped RNA. As the ratio of the cap analog to GTP increases in the reaction, the ratio of capped to uncapped RNA increases proportionally. Increasing the ratio of cap analog to GTP in the transcription reaction produces lower yields of total RNA because the concentration of GTP becomes limiting when holding the total concentration of cap and GTP constant. Thus, the final RNA yield is dependent on GTP concentration, which is necessary for the elongation of the transcript. Once it is used up, then the reaction terminates. The other nucleotides (ATP, CTP, UTP) are present in excess at 7.5 mM in a mMESSAGEmMACHINE® reaction.

There are two reasons why the total concentration of cap and GTP (at a 4:1 ratio) are not increased to increase yields. First, cap analog is very expensive and second, higher nucleotide concentrations in the transcription reaction can be inhibitory. In this strategy, the GTP concentration is limiting and the yield is not as high as in a reaction where the GTP concentration is equal to the other nucleotides. Generally, a mMESSAGEmMACHINE® capping reaction will yield 1 mg/ml of reaction product. If one considers that a non-capping reaction can generate up to 8 mg/ml of RNA, then the potential for much greater yields of capped RNA is possible if a strategy is developed to overcome the limiting GTP concentration.

Capped RNA encoding specific genes can be transfected into eukaryotic cells or microinjected into cells or embryos to study the effect of translated product in the cell or embryo. If uncapped RNA is used in these experiments, the RNA is degraded quickly and very little protein is translated from the in vitro transcribed, capped RNA.

In more recent years, the use of capped RNA for therapeutic purposes has been studied. Mainly, it has the potential to be used to generate vaccines against infectious diseases or cancers (Sullenger, 2002). Capped RNA is used to produce non-infectious particles of Venezuelan Equine Encephalitis virus containing an RNA encoding an immunogen. These non-replicating viral particles are injected into humans where they can enter host cells. Once in the host cell, the viral particle dissociates and the mRNA encoding the immunogen is translated into protein. These proteins can induce an immune response. These types of vaccines are in development for human immunodeficiency virus (HIV), feline immunodeficiency virus, human papillomavirus type 16 tumors, lassa virus, ebola virus, marburg virus, anthrax and botulinum toxin (Burkhard, 2002; Davis, 2002; Eiben, 2002; Geisbert, 2002; Hevey, 1998; Pushko, 1997; Pushko, 2000; Lee, 2001; Lee, 2003).

Another approach in use is to isolate dendritic cells from a patient and then to transfect the dendritic cells with capped RNA encoding an immunogen. The dendritic cells translate the capped RNA into a protein that induces an immune response against this protein. In a small human study, immunotherapy with dendritic cells loaded with CEA capped RNA was shown to be safe and feasible for pancreatic cancer patients (Morse, 2002). It was also noted that introducing a single capped RNA species into immature dendritic cells induced a specific T-cell response (Heiser, 2002).

These vaccine strategies will require large quantities of capped RNA. Developing methods to synthesize and purify capped RNA will be important to make these vaccines commercially feasible. As well, strategies to increase the percentage of full-length capped RNA in a transcription reaction leading to a more homogenous product will be preferred in the vaccine industry as highly pure components are usually required for human use. In addition, researchers prefer to use products that are as pure as possible to minimize the number of variables in an experiment. As well, the purer the product, the more potent it is. Current protocols, enabling the production of about 1 mg/ml of capped RNA, are simply insufficient for the scale of production needed for these applications.

Thus, new or improved methods and compositions are needed for increasing the yield of usable, translatable RNA, while keeping costs at a minimum. Moreover, such methods and compositions that are generally applicable to reactions involving competing reactants are desirable.

### SUMMARY OF THE INVENTION

The present invention concerns methods and compositions for obtaining concentrations of capped transcripts higher than were previously attainable. In specific embodiments, the methods and compositions of the invention enable more capped full-length RNA to be produced from a transcription and capping reaction because they overcome problems associated with the changes in concentration of nucleotides that compete with a cap structure, relative to the concentrations of that cap structure. These problems are overcome by supplementing particularly the concentration of GTP, which competes with the cap structure, so as to prevent the GTP from being concentration-limiting in the reaction. It will be understood that the term “capped transcript” refers to a full-length transcript that is capped, unless otherwise specifically indicated. Transcripts are RNA molecules, and thus, the terms “capped transcript” and “capped RNA” are used interchangeably herein. The term “capped” means that there is a cap structure at the 5′ end of the transcript. The term “cap structure” refers to a chemical structure represented as m7G (7-methylguanosine) where the m7G is bonded to the 5′ triphosphate of the first nucleotide of the transcript through its 5′-hydroxyl group to produce the structure m7GpppN.

Moreover, the invention can be applied more generally to the incorporation of any nonextending nucleotide into an RNA molecule during a transcription reaction. In specific embodiments, at its 5′ end the transcript has a nonextending nucleotide with cap functionality, while in others the nonextending nucleotide does not have cap functionality. It is contemplated that a cap analog can be employed as the nonextending nucleotide with cap functionality.

Therefore, the present invention includes methods for producing capped RNA from a capping and transcription reaction with increased yield and/or methods for producing capped RNA from a capping and transcription reaction involving lower amounts of a cap analog relative to the yield. The present invention enables the production of capped RNA in concentrations greater than was previously obtained. Thus, embodiments of the invention include where the reaction yield of capped RNA produced is about, is at least about, or is at most about 1, 2, 3, 4, 5, 6, 7, 8, 9, 10, 11, 12, 13, 14, 15, or more mg/ml, or any range derivable therein. The term “reaction yield” refers to the concentration of reaction product before any isolation or purification steps are taken. In specific cases, between about 1 mg/ml and about 10 mg/ml or between about 4 mg/ml and about 7 mg/ml is the reaction yield concentration of capped transcript.

In certain aspects of the invention, methods for producing capped RNA are provided in which at least the following steps are employed: a) incubating components for a transcription and capping reaction under conditions to promote transcription and capping, wherein the components include a cap analog, a nucleotide that competes with the cap analog, and non-competing nucleotides; and, b) supplementing the reaction with the competing nucleotide to maintain the concentration of the competing nucleotide in the reaction at a ratio between about 1:1 and about 1:50 relative to the concentration of the cap analog in the reaction. The term “incubating” in conjunction with a “reaction” is used according to its ordinary and plain meaning in the field of molecule biology to refer to “mixing together components and maintaining the reaction under given conditions in a controlled or artificial environment.” The term “supplementing” is used according to its plain and ordinary meaning, which is “providing to make up for a deficiency.” In the context of methods of the invention, a reaction component is supplemented by adding that component to the reaction after the reaction has begun.

Methods of the invention generally involve providing a relatively low concentration of the nucleotide that competes with the cap analog and adding the competing nucleotide at least one time after an initial batch reaction or continuously during the reaction. The “relatively low concentration” is relative to the concentration of the cap analog in the reaction. Thus, embodiments of the invention involve keeping the amount of the competing nucleotide in the reaction within a desirable range or below a certain level by limited supplementation of that competing nucleotide so as to allow the reaction product to be efficiently produced. Moreover, in embodiments of the invention, the concentration of the competing nucleotide is relative to the amount of a cap analog in the reaction. This can be expressed as a ratio between the concentration of the competing nucleotide in the reaction and the concentration of the cap analog in the reaction.

In various methods of the invention, GTP may be specifically used in the reaction. The method does not depend on whether GTP or a GTP analog is used, so long as the analog is incorporated at a rate similar to GTP by the polymerase into the elongated transcript. Of course, the term “GTP analog” or the analog of any other extending nucleotide (that is, nucleotides that can be incorporated into the growing transcript at any position) is not meant to refer to a cap analog, unless a cap analog is specifically designated, or to a compound that is a non-extending nucleotide (incapable of being incorporated into a growing transcript at any position).

In other embodiments of the invention, a nucleotide other than GTP is used in methods and kits of the invention when that nucleotide competes with a cap analog in the transcript. In certain cases, the nucleotide is ATP or an ATP analog. As discussed earlier, an A has been observed in the +1 site of a T7 promoter. It will be understood that any embodiment discussed with respect to GTP or a GTP analog may be similarly implemented with ATP or an ATP analog.

The phrase “transcription and capping reaction” will be understood to refer to a reaction in which capped transcripts are produced. Furthermore, a transcription and capping reaction will be understood to contain at least an enzyme that polymerizes the transcript, incorporated nucleotides (or nucleotide analogs), and a cap analog. Such a reaction will typically include nucleotides (or nucleotide analogs), an RNA polymerase, a cap analog, and appropriate buffers and/or salts.

The term “cap analog” refers to a non-extendible di-nucleotide that has cap functionality (facilitates translation or localization, and/or prevents degradation of the transcript) when incorporated at the 5′ end of a transcript, typically having an m7GpppG or m7GpppA structure. A cap analog is specifically contemplated for use with the invention. Unless otherwise indicated, the term “reaction” is used to refer to a single reaction. While it is contemplated that one or more components of a reaction may be supplemented during a single reaction, when all of the components have been supplemented into the reaction, it is no longer the same reaction. Moreover, in some embodiments, the reaction does not include the supplementation of polymerase after the initial reaction mixture is created.

Typically, because the reaction is not supplemented with a cap analog in some embodiments of the invention, the concentration range of the competing nucleotide depends on the initial concentration of the cap analog. In particular embodiments, the concentration of a competing nucleotide in the reaction is expressed as a ratio relative to the initial concentration of the cap analog or non-extending nucleotide in the reaction. Ratios implemented with respect to the invention are between about 1:1 and about 1:50, though it is contemplated to be about, at least about, or at most about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, or more, or any range derivable therein. The term “initial concentration” is understood to mean the concentration of a component at the start of the reaction. The start of the reaction is when the reaction begins (i.e., polymerization), after all of the components necessary for the reaction are incubated together. For a transcription and capping reaction, the compound that provides the cap structure is one of the necessary components of the reaction.

In embodiments in which the concentration of the competing nucleotide is maintained or introduced at a “relatively low level” compared to the concentration of a cap analog in the reaction, it will be understood that this means that the ratio of the concentration of the competing nucleotide to the concentration of a cap analog is about or less than about 1:10 or any lower ratio—such as 1:20—as discussed in the previous paragraph.

Maintaining the relatively low level of concentration of the competing nucleotide in the reaction can be achieved by a number of ways. It is contemplated that supplementation of components may be implemented through supplementation that is continuous, periodic, or intermittent, or a combination thereof.

In many embodiments of the invention, this is achieved by a fed-batch process. The term “fed-batch process” means that there is an initial reaction mixture in which all of the components are present (batch reaction) and that the reaction is then occasionally supplemented with one or more components thereafter. Thus, a component introduced by the fed-batch process refers to the supplementation of that component in discrete amounts to a reaction after the reaction has commenced. However, the invention is not contemplated as limited to supplementation by a fed-batch process. Any embodiment employing a fed-batch process can be implemented with respect to other supplementation procedures, such as continuous flow, and vice versa.

With a capping and transcription reaction, for example, the reaction commences when an RNA polymerase mediates the formation of a covalent bond between a nucleotide and a cap analog. It will be understood that the difference between a capping and transcription reaction and just a transcription reaction is the presence of a component that provides the cap structure to the 5′ end of a transcript.

The commencement of the reaction may proceed from a batch reaction, which means that all of the reaction components required for the reaction to begin are initially incubated together. Thereafter, in embodiments of the invention, supplementation of one or more of the same or different components of the reaction is part of the methods of the invention.

In certain embodiments of the invention, methods involve supplementing a transcription and capping reaction with GTP or a GTP analog because it competes with a cap analog in certain reactions, such as when T7, SP6 or T3 polymerase is used to catalyze the reaction. It will be understood, however, that the invention is not limited to GTP or a GTP analog. Instead, the invention can be implemented with respect to any reaction involving a nucleotide that competes with a cap analog or other nonextending mono- or di-nucleotide that can be incorporated at the 5′ end of the transcript. Thus, it is specifically contemplated that any embodiment involving GTP or a GTP analog as the competing nucleotide can be implemented with respect to a different nucleotide or nucleotide analog. The term “nonextending nucleotide” means a nucleotide that 1) does not have a 5′ triphosphate or has a 5′ triphosphate that has been modifed, both of which allow the nucleotide to be incorporated only at the 5′ end of a transcript, and 2) has a 3′ hydroxy, so it can be extended at the 3′ position. In specific embodiments, the nonextending nucleotide is a mono- or di-nucleotide, meaning it has a single or double nucleotide structure. These nucleotides may or may not have cap functionality. Cap analogs are examples of nonextending di-nucleotides having cap functionality.

While reaction components may be added to the reaction continuously, in some embodiments of the invention, one or more competing components is provided to the reaction by a fed-batch process. The fed-batch process is used, in some embodiments of the invention, to supplement a reaction with one or more reaction components. In specific embodiments, a component is supplemented to the reaction by a fed-batch process periodically or intermittently. The term “periodically” is used to mean “occurring at regular intervals,” with “regular” understood to mean “fixed” with respect to some characteristic, such as time or concentration level in the reaction of a supplemented component. The term “intermittently” is used to mean “occurring at intervals,” though the intervals are not necessarily regular. It will be understood that “intermittent” introduction of a reaction component can also be “periodic.” It will further be understood that intermittent introduction or supplementation of a component to a reaction means at least one time, while “periodic” introduction or supplementation of a component is at least two times (to define the “regular interval”). It is contemplated that a component may be supplemented, supplemented at least, or supplemented at most 1, 2, 3, 4, 5, 6, 7, 8, 9, 10, 15, 20, 25, 30, 35, 40, 45, 50, 60, 70, 80, 90, 100 or more times, or any range derivable therein, during the course of a single reaction.

Thus, embodiments of the invention involve introducing to a transcription and capping reaction GTP or a GTP analog by a fed-batch process. In some embodiments, it is specifically contemplated that GTP or a GTP analog is provided the reaction at least twice. In further embodiments, it is contemplated that GTP or a GTP analog is introduced intermittently or periodically into the reaction between three times and 50 times. Any embodiments discussed with respect to a fed-batch process may be implemented more generally as part of the invention so long as one or more components are supplemented, regardless of how this is achieved.

It is contemplated that supplementation of a reaction is discrete in that components are added to the reaction, but not exchanged. Thus, a fed-batch process is not understood as involving a continuous exchange of reaction components and/or reaction byproducts.

In certain embodiments of the invention, methods involve supplementing a transcription and capping reaction with GTP or a GTP analog because it competes with a cap analog in certain reactions, such as when T7, SP6 or T3 polymerase is used to catalyze the reaction. It will be understood, however, that the invention is not limited to GTP or a GTP analog. Instead, the invention can be implemented with respect to any reaction involving a nucleotide that competes with a cap analog or a nonextending mono- or di-nucleotide that can be incorporated at the 5′ end of the transcript. Thus, it is specifically contemplated that any embodiment involving GTP or a GTP analog as the competing nucleotide can be implemented with respect to a different nucleotide or nucleotide analog.

GTP or a GTP analog is supplemented into a reaction in many embodiments of the invention. In certain embodiments, this is achieved by a fed-batch process. In any method of the invention, GTP may be specifically used in the reaction. The method does not depend on whether GTP and/or a GTP analog are used, so long as the analog is incorporated at a rate similar to GTP by the polymerase into the elongated transcript. Of course, the term “GTP analog,” as used herein, refers to extending nucleotides, and thus, excludes any cap analogs, as defined below.

Other methods are included for increasing the yield of capped full-length RNA transcript comprising: incubating components for a transcription and capping reaction under conditions to promote polymerization of the transcript, wherein the concentration of a cap analog is maintained in the reaction at a ratio of between about 1:1 and about 50:1 relative to the concentration of a competing nucleotide component by multiple administration of the competing nucleotide component. In specific embodiments, the competing nucleotide is GTP or a GTP analog. In reactions involving T7, T3, or SP6 RNA polymerase, the competing nucleotide is typically GTP, or analogs thereof. It is specifically contemplated that any embodiment involving the use of GTP or a GTP analog may be substituted with another nucleotide or nucleotide analog when using an RNA polymerase that employs that particular nucleotide at the +1 position.

The present invention also concerns methods for increasing the yield of capped transcripts in an in vitro transcription and capping reaction comprising: incubating reaction components under conditions that enable transcription, wherein the concentration of GTP or a GTP analog in the reaction is maintained at a concentration between about 0.2 mM and about 2.0 mM and the concentration of other nucleotides is at least about 0.2 mM for at least 30 minutes during the reaction.

Moreover, the present invention involves methods for producing RNA with a non-extending nucleotide at the 5′ end comprising introducing a nucleotide that competes with the non-extending nucleotide by a fed-batch process to a transcription reaction comprising RNA polymerase and the non-extending nucleotide. In particular embodiments, the non-extending nucleotide is not a cap analog from a functional standpoint. It is specifically contemplated that any embodiment discussed with respect to GTP or a GTP analog may be implemented with respect to another nucleotide so long as that nucleotide competes with a non-extending nucleotide at the 5′ end, and vice versa. Furthermore, it will also be understood that any embodiment discussed with respect to a cap analog can be implemented with respect to a non-extending nucleotide capable of being added only to the 5′ end of the transcript, and vice versa.

In some methods of the invention, the nucleotide incorporated into the growing transcript that effectively competes with the 5′ non-extending nucleotide is provided to the reaction by a fed-batch process. Though in particular embodiments a GTP or GTP analog is added by a fed-batch process, other components of a capping/transcription reaction may also be introduced by the fed-batch process. However, it is contemplated that in some embodiments of the invention, a cap analog is not added by a fed-batch process. Under these circumstances, this will be understood to mean that no more than 1% of the total amount of cap analog is supplemented, for example, by a fed-batch process (which means that at least trace, contaminating, and/or minute amounts of cap analog cannot be supplemented as a way around the invention). It certain embodiments, the reaction can be supplemented with a cap analog.

In some embodiments of the invention one of the components introduced to the reaction by the fed-batch process is a nucleotide. In some cases, more than one nucleotide is introduced by the fed-batch process. For example, both GTP and CTP nucleotides may be introduced by a fed-batch process, or GTP and a GTP analog may be introduced by a fed-batch process. In further embodiments, all of the nucleotides are introduced by a fed-batch process. One or more of the nucleotides in the reaction may be a modified nucleotide. Non-cap nucleotides may be modified but still be functional in that they may be incorporated at the 3′ end onto a polymerized transcript; that is, these non-cap modified nucleotides are extendable because they have a 5′ triphosphate.

In specific embodiments, the nucleotide introduced by the fed-batch process is GTP and/or a non-cap GTP analog. A “GTP analog” will be understood as referring to a GTP analog that does not have “cap structure” as described above (that is, it is not a cap analog). Furthermore, the phrase “GTP or GTP analog” means GTP and/or GTP analog; moreover, any concentration referring to a GTP or GTP analog means the concentration of GTP or GTP analog, unless both are present, in which case it refers to the concentration of GTP and GTP analog. In some instances, the concentration of GTP or a GTP analog introduced into the reaction by a fed-batch process depends on the concentration of a cap analog in the reaction. In some cases, the concentration of GTP or GTP analog introduced into the reaction depends on the initial concentration of a cap analog in the reaction. In some embodiments, the concentration of GTP introduced into the reaction is determined based on the ratio of the concentration of GTP in the reaction after the GTP is introduced to the initial concentration of the cap analog in the reaction.

The initial concentration of GTP (and/or GTP analog) in the reaction is contemplated to be about or at most about 0.01, 0.05, 0.1, 0.15, 0.20, 0.25. 0.30, 0.35, 0.40, 0.45, 0.50, 0.55, 0.60, 0.65, 0.70. 0.75, 0.80, 0.85, 0.90, 0.95, 1.0, 1.25, 1.50, 1.75, 2.0, 2.25, 2.5, 2.75, 3.0, 3.25, 3.5, 3.75, 4.0 or more mM, or any range derivable therein. In specific embodiments, the initial concentration of GTP or GTP analog in the reaction is about or less than about 1.0 mM. The initial concentration of GTP or GTP analog may be introduced using the same device to implement the fed-batch process, or it may not; such as when the reaction starts off as a batch reaction. Thereafter, in some embodiments, the amount of GTP or a GTP analog introduced in the reaction (this is, the supplementation step) increases the concentration of GTP or GTP analog in the reaction by about or less than about 0.05, 0.1, 0.15, 0.20, 0.25. 0.30, 0.35, 0.40, 0.45, 0.50, 0.55, 0.60, 0.65, 0.70. 0.75, 0.80, 0.85, 0.90, 0.95, 1.0, 1.25, 1.50, 1.75, 2.0, 2.25, 2.5, 2.75, 3.0, 3.25, 3.5, 3.75, 4.0 or more mM, or any range derivable therein, overall or with respect to each introduction or supplementation. In particular embodiments, the amount of GTP or a GTP analog introduced in the reaction by the fed-batch process increases the concentration of GTP or GTP analog in the reaction by between about 0.1 mM and about 2.0 mM. In still further embodiments, the amount of GTP or a GTP analog introduced in the reaction by the fed-batch process increases the concentration of GTP or GTP analog in the reaction by between about 0.25 mM and about 0.5 mM.

The initial concentration of cap analog in the reaction is about, at least about, or at most about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15 or mM, or any range derivable therein. In specific embodiments, the initial cap analog concentration is between about 1 mM and about 10 mM or between about 2 mM and about 6 mM.

In some embodiments of the invention, the initial concentrations of each of the other nucleotides in the reaction (C, A, and U when GTP is the nucleotide that competes for the cap analog) is about, at least about, or at most about 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15 or mM, or any range derivable therein. In certain embodiments, the initial concentration of each of the other nucleotides in the reaction is between about 1 mM and about 10 mM. It is contemplated that the concentration of other nucleotides may be the same for each other nucleotide, or they may be different. The concentration of one or more of the other nucleotides may or may not be dependent on the concentration of the nucleotide that competes with a cap analog in the reaction. In certain embodiments, the concentration of one of the other nucleotides is dependent on the amount of that competing nucleotide (or vice versa). In some embodiments, the ratio between the initial concentration of the competing nucleotide and one of the other nucleotides in the reaction is about, at least about, or at most about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25 or more, or any range derivable therein.

The initial reaction volume can vary. In certain embodiments of the invention, the initial reaction volume is about, at least about, or at most about 0.010, 0.020, 0.030, 0.040, 0.050, 0.060, 0.070, 0.080, 0.090, 0.010, 0.15, 0.020, 0.025, 0.030, 0.035, 0.040, 0.045, 0.050, 0.055, 0.060, 650, 0.070, 0.075, 0.080, 0.085, 0.090, 0.095, 0.100, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10 or more ml, or any ranger derivable therein. In specific embodiments, the initial reaction volume is between about 10 μl and about 10 ml, while in others it is at least about 100 μl or at least about 1 ml.

While recognizing that concentration is dependent on volume, the inventors further contemplate that the volume added to the reaction by the fed batch process can be important. Thus, in some embodiments of the invention, the volume added is between about 0.1 μl and about 10 ml. In certain embodiments of the invention, the volume of one or more components added intermittently or periodically by a fed batch process—that is, each time a component is added—is about or at most about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 21, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900 μl or ml, or more, or any range derivable therein. The total volume added by a fed-batch process includes the volumes and ranges of volumes in the previous sentence and further may be about or at most about 110, 120, 130, 140, 150, 160, 170, 180, 190, 200 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 μl or ml, or more, or any range derivable therein and from above. Alternatively, the volume added by a fed-batch process can be referred to in terms of the reaction volume. Thus, in some embodiments, the volume introduced intermittently or periodically to the reaction is about or less than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.5, 7.0, 7.5, 8.0, 8.5, 9.0, 9.5, or 10.0%, or any range derivable therein, of the total reaction volume at the time the volume is added by a fed-batch process.

In certain embodiments, a reaction component is provided to a reaction continuously. It is understood that “continuous” supplementation means that a component is provided to the reaction throughout the entire reaction or at least throughout the duration of the reaction during which the rate for producing the reaction product is maximal. Continuous supplementation involves supplementation of one or more components at a constant flow rate in some embodiments of the invention, while in others the flow rate of one or more components can change during the reaction. In embodiments, where the competing nucleotide is provided continuously, it is contemplated that it can be continuously added to the reaction at a rate of between about 10 μM per minute to about 200 μM per minute. It is contemplated that the rate of component or components added continuously to the reaction is about or at most about 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300 μM or more per minute, or any range derivable therein.

The reaction may be allowed to proceed for any length of time, though it is particularly contemplated that the reaction will be allowed to proceed as long as polymerization is occurring. That length of time will be dependent on factors such as concentration and longevity of enzyme, degradation factors, temperature, volume, and concentration of other reaction components. In certain embodiments, the reaction time (refers to the length of time between when a single reaction starts and when the reaction is terminated or stops) is about, at least about, or at most about 1, 2, 3, 4, 5, 6, 7, 8, 9, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 minutes or more, as well as about 1, 2, 3, 4, 5, 6, 7, 8, 9, 10, 11, 12, 13, 14, 15, 16, 17, 18, 19, 20, 21, 22, 23, 24 hours or more, or any range derivable therein.

It is thus contemplated that methods and compositions of the invention can be employed to obtain a higher yield of reaction product from one or more reactions. The invention, in some embodiments, allows for an increase in yield that is about or at least about 110%, 120%, 130%, 140%, 150%, 160%, 170%, 180%, 190%, 200%, 210%, 220%, 225%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500% or more, or any range therein compared to yields obtained from reactions involving the same or similar initial concentrations of competing reaction components. Alternatively, the increase in desired reaction product may be about or at least about 2-, 3-, 4-, 5-, 6-, 7-, 8-, 9-, 10-, 15-, 20-, 25-fold or more, or any range therein, as compared to the amount achieved when methods and/or compositions of the invention are not employed.

The present invention concerns methods in which one of the components introduced to the reaction by a fed-batch process is a cap analog. Cap analogs include, but are not limited to, a chemical structure selected from the group consisting of m7GpppG, m7GpppA, m7GpppC; unmethylated cap analogs (e.g., GpppG); dimethylated cap analog (e.g., m2,7GpppG), trimethylated cap analog (e.g., m2,2,7GpppG), dimethylated symmetrical cap analogs (e.g., m7Gpppm7G), or anti reverse cap analogs (e.g., ARCA; m7,2′OmeGpppG, m72′dGpppG, m7,3′OmeGpppG, m7,3′dGpppG and their tetraphosphate derivatives) (Stepinski et al, 2001; Jemielty et al., 2003, which are hereby incorporated by reference). The present invention particularly includes a method for producing capped RNA comprising introducing at least GTP by a fed-batch process to a solution comprising components for a transcription and capping reaction, wherein the reaction comprises RNA polymerase, nucleotides, a cap analog.

In embodiments of the invention, other components of the transcription and capping reactions include pyrophosphatase, a magnesium salt, one or more modified non-cap nucleotides, RNA polymerase, ribonuclease inhibitor, or an enzyme for generating utilizable nucleotides (that is, precursor nucleotides are mixed in the reaction but they are processed by the enzyme to render them useable in the transcription and capping reaction). In specific embodiments, the salt is a magnesium salt. It is contemplated that any of these other components may be introduced by themselves or in combination with one or more other components by a fed-batch process.

It is contemplated that any template may be employed in the transcription and capping reactions, though the use of a template encoding viral transcripts and transcripts encoding immunogens from pathogens is specifically contemplated.

In some embodiments, the fed-batch process in implemented by the use of an electronic device, which may or may not be programmed to administer components to the reaction at particular times or when the concentration of a component reaches or is expected to reach a particular level or range. In some cases, the fed-batch process involves not an electronic device but manual administration. One or more components may be added to the reaction at a certain time or when the concentration of a component is expected to reach a particular level or range. It will be understood that the invention is not focused on the specific way in which components are added to the reaction but that in some embodiments, that way is identified.

In methods of the invention, one or more reaction components may be immobilized, meaning that the component is unable to move freely in solution, such as being physically attached to a structure. In particular embodiments, the template is immobilized. In other embodiments, the component is immobilized using a non-reacting structure. For example, the component may be attached to the non-reacting structure, which refers to a structure that is not involved in the reaction. The non-reacting structure may be composed of plastic, metal, or glass. In some cases, it has the shape of a column or bead, or a membrane is involved. In specific embodiments, the non-reacting structure has streptavidin or cellulose, such as a streptavidin bead.

It is contemplated that the fed-batch process may be implemented through use of a manual device. The manual device may introduce one or more reaction components to the reaction one or more times. It will be understood that a manual device refers to a device operated directly and manually by a person. Alternatively, the fed-batch process may be implemented through use of an electronic device. In some embodiments, the electronic device is programmed to introduce one or more reaction components. In further embodiments, the fed-batch process involves an electronic device that maintains the concentration of the introduced component(s) in the reaction for a certain length of time. Moreover, in other embodiments, the fed-batch process involves an electronic device that periodically increases the concentration of the introduced components in the reaction. The concentration may be increased 1, 2, 3, 4, 5, 6, 7, 8, 9, 10 or more times during a single reaction. An electronic device may employ a syringe to deliver a component; furthermore, more than one syringe may be employed in the process.

It is contemplated that each or all components added to the reaction by a fed-batch process may be delivered in a volume of between about 0.1 μl and about 100 μl. The volume may be about, at least about, or at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more microliters, or any range derivable therein.

The total amount of capped RNA produced may be in terms of the amount of reaction product from a single reaction (that is, prior to any pooling). In embodiments of the invention, the amount of capped RNA transcripts produced from a single reaction is about, at least about, or at most about 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 1100, 1200, 1300, 1400, 1500, 1600, 1700, 1800, 1900, 2000 or more milligrams (mg) or grams (g), or any range derivable therein.

In some embodiments, methods for large scale production are provided. The term “large-scale production” refers to reaction yield of reaction product on the order of milligram quantities of at least about 1 g. In some embodiments, there are methods for large scale production of capped transcripts comprising introducing GTP or a GTP analog by a fed-batch process to a reaction mixture comprising RNA polymerase, ribonucleotides, and a cap analog, wherein at least about 1 gram of capped full-length RNA transcripts are produced.

The present invention also concerns compositions that can be used in methods of the invention or to implement methods of the invention. Kits for producing a reaction product that involves competing components are part of the invention. Particularly contemplated is a kit for producing capped RNA comprising RNA polymerase, nucleotides, and a cap analog. In certain embodiments, a kit also includes a ribonuclease inhibitor. Buffers can be included in any kit of the invention, including enzyme buffer and nucleotide buffer.

Solutions used with methods of the invention may be added in a concentrated form or they may be provided in kits in a concentrated form. The solutions may be 2×, 3×, 4×, 5×, 10×, or 20×.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

It is specifically contemplated that any embodiments described in the Examples section are included as an embodiment of the invention.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a graph showing the yield of a standard mMESSAGE mMACHINE (mMmM) reaction over time. The p4kb template was transcribed in 6 replicate standard mMmM reactions (20 μl, 6 mM m7GpppG, 1.5 mM GTP). After the indicated incubation times, reaction products were DNase I-treated, purified on a glass fiber column, and quantified by UV spectrophotometry.

FIG. 2 is bar graph showing the variation in yield and capping efficiency when CAP or GTP concentration is changed in a standard mMESSAGE mMACHINE reaction. The p4kb template was transcribed in a standard mMmM reaction (6 mM CAP, 1.5 mM GTP) or in the presence of 12 mM cap (2× CAP) or 3.75 mM GTP (2.5× GTP). The graph shows the % variation respectively to the standard mMmM reaction. To reflect the fact that 100% capping is a maximal theoretical limit, the % variation capping is calculated as follows: % variation capping=[(1-mMmM capping)/(1-experimental capping)]×100. % variation yield=(experimental yield/mMmM yield)×100.

FIG. 3 is bar graph showing the yield of transcription reactions with the p4kb template. Standard mMmM reactions without or with 1 to 4 additions of 20 nmol GTP every 30 min (at 30, 60, 90 and 120 min) were performed. All the reactions (20 μl) were incubated for 150 min at 37° C. and quantified after DNAse I treatment and purification on glass fiber column. Experiment was performed in duplicate.

FIG. 4 is bar graph showing the variation in yield and capping efficiency for transcripts prepared with a mMmM reaction in the presence of the ARCA m7,3′-OMeGpppG (6 mM) or two fed-batch reactions. GTP was either fed by 0.5 mM increment every 15 min for 1 hour (FB1, 2 mM added) or by 1 mM increment every 30 min for 2 hours (FB2, 4 mM added). Transcription reactions (20 μl) were performed with the p4Kb template, incubated for 2.5 hours at 37° C. and analyzed as in FIG. 2.

FIG. 5 is bar graph showing the variation in yield and luciferase activity for transcripts prepared with a mMmM reaction in the presence of the ARCA m7,3′-OMeGpppG or two fed-batch reactions. Transcription reactions (20 μl) were performed as described in FIG. 4 with the pAmbluc template. Each capped luciferase mRNA (0.4 μg) were transfected in 1×10⁵ HeLa cells in triplicate and luciferase activity was analyzed 18 hours after transfection. % variation luc activity=(experimental luc activity/mMmM luc activity)×100.

FIG. 6 is bar graph showing the variation in yield and capping efficiency for transcripts prepared with a standard mMmM, 3 fed-batch reactions with 2, 5 or 7 additions of 10 nmol GTP every 15 min and 2 control batch reactions with performed with 3 mM cap analog and 4 or 1.5 mM GTP. Transcription reactions (20 μl) were performed with the p4Kb template, incubated for 2.5 hours at 37° C. and analyzed as in FIG. 2.

FIG. 7 is bar graph showing the variation in yield and capping efficiency for transcripts prepared with a standard mMmM, a mMmM performed with 3 mM cap analog and 6.5 mM GTP or 2 different fed-batch reactions. Both fed-batch reactions contained an initial concentration of 3 mM cap analog and GTP addition was performed by a computer-controlled Hamilton 540B syringe pump. GTP was either fed by 0.5 mM increment (0.5 μl at 100 mM) every 10 min in a reaction started with 0.5 mM GTP (FB1) or by 0.25 mM increment (0.25 μl at 100 mM) every 5 min in a reaction started with 0.25 mM GTP (FB2). Transcription reactions (100 μl) were performed with the p4Kb template, incubated for 2.5 hours at 37° C. with constant homogenization using a magnetic stir bar and analyzed as in FIG. 2.

FIG. 8 shows electropherograms of purified transcripts analyzed on a RNA Nano LabChip® with and Agilent™ 2100 bioanalyzer. 1.25 μg of 5′ biotinylated PCR product immobilized on strepdavidin beads was used in three successive fed-batch reactions using the Hamilton 540B syringe pump and the FB2 method described in FIG. 7. Between each round, the beads were spun down, the supernatant pulled out for subsequent purification and analysis, and fresh transcription components were added to the beads. PCR product (1.7 kb) was prepared from the pAmbluc template, cleaned up with DNAclear™ (Ambion) and bound to Power-Bind™ Strepdavidin beads (Seradyn) as recommended by the manufacturer.

### DESCRIPTION OF ILLUSTRATIVE EMBODIMENTS

The present invention overcomes the deficiencies of current procedures of obtaining a high yield of reaction products when there is at least one limiting reagent in the reaction that competes with another reaction component. Moreover, the invention accomplishes this in a way that is cost efficient.

I. Transcription and Capping Reactions

The present invention can be implemented with respect to any transcription reaction involving competing components, particularly when one of the competing components can become yield-limiting, or when one of the competing components is relatively expensive compared to other competing components, and/or when both situations are present.

A reaction in which transcribed RNA is capped provides such an example. In vitro transcription reactions are well known to those of skill in the art. Protocols and conditions for such reactions can be found, for example, in Sambrook et al. 2001; Sambrook et al., 1989; Ausubel, 1996; and, U.S. Pat. No. 5,256,555, all of which are hereby incorporated by reference in their entireties. Kits for such reactions are also widely available and their protocols can be readily obtained, for example, Ambion's MEGAscript® High Yield Transcription Kit, Ambion's MEGAshortscript® High Yield Transcription Kit, and Ambion's mMESSAGE mMACHINE® High Yield Capped RNA Transcription Kit.

A. Template Preparation

Typically, in vitro transcription requires a purified, linear DNA template containing a promoter, ribonucleotide triphosphates, a buffer system that includes dithiothreitol (DTT) and magnesium, and an appropriate RNA polymerase. The exact conditions used in the transcription reaction depend on the amount of RNA needed for a specific application.

Common RNA polymerases used in in vitro transcription reactions are SP6, T7 and T3 polymerases, named for the bacteriophages from which they were cloned. The genes for these proteins have been overexpressed in Escherichia coli and the polymerases have been rigorously purified. RNA polymerases are DNA template-dependent with distinct and specific, consensus promoter sequence requirements, which are well known in the art. After the RNA polymerase binds to its double-stranded DNA promoter, the polymerase separates the two DNA strands and uses the 3′ to 5′ strand as the template for the synthesis of a complementary 5′ to 3′ RNA strand. Depending on the orientation of the DNA sequence relative to the promoter, the template may be designed to produce sense strand or antisense strand RNA.

When the common phage polymerases are employed, the DNA template must contain a double-stranded promoter region where the phage polymerase binds and initiates RNA synthesis.

Most transcription templates used in the laboratory are plasmid constructs engineered by either cloning or PCR. Many common plasmid cloning vectors include phage polymerase promoters, and they often contain two distinct promoters—one flanking each side of the multiple cloning site, allowing transcription of either strand of an inserted sequence. Plasmid vectors used as transcription templates should be linearized by restriction enzyme digestion. Because transcription proceeds to the end of the DNA template, linearization ensures that RNA transcripts of a defined length and sequence are generated. The restriction site need not be unique as long as the promoter remains adjacent to the sequence to be transcribed; the vector itself may be digested multiple times. It is unnecessary to purify the promoter-insert sequence away from other fragments prior to transcription because only those fragments containing the promoter sequence will serve as template. It is recommended, though not always required, that restriction enzyme digestion should be followed by purification (e.g., phenol:chloroform extraction, Sephadex® G-50 column) because contaminants in the digestion reaction may inhibit transcription.

PCR products can also function as templates for transcription. A promoter can be added to the PCR product by including the promoter sequence at the 5′ end of either the forward or reverse PCR primer. These bases become a double-stranded promoter sequence during the PCR reaction. Also, two oligonucleotides can be used to create short transcription templates. Two complementary oligonucleotides containing a phage promoter sequence, are simply annealed to make a double-stranded DNA template. Only part of the DNA template (the −17 to +1 bases of the RNA polymerase promoter) needs to be double-stranded. It may be more economical, therefore, to synthesize one short and one long oligonucleotide, generating an asymmetric hybrid.

When designing a transcription template, it must be decided whether sense or antisense transcripts are needed. Sense strand transcripts are used when performing expression, structural or functional studies or when constructing a standard curve for RNA quantitation using an artificial sense strand RNA. By convention, the single strand of a DNA sequence shown in scientific journals and databases, is the coding, (+), or “sense strand”, identical in sequence (with T's changed to U's) to its mRNA copy. The mRNA then serves as a template for translation. Its 5′ or upstream sequence contains the AUG which corresponds to the NH₃-terminal methionine of the protein. The +1 G of the RNA polymerase promoter sequence in the DNA template is the first base incorporated into the transcription product. To make sense RNA, the 5′ end of the coding strand must be adjacent to, or just downstream of, the +1 G of the promoter.

B. In vitro Transcription and Capping Reactions

The MEGAscript® family of kits use Ambion's high yield technology to synthesize RNA for applications where large mass amounts are required. High nucleotide concentration (7.5 mM each) and optimized reaction condition allow yields up to 8 mg/ml.

However, in certain applications, capped RNA is desirable. In eukaryotes, mRNA (transcribed by RNA polymerase II) is capped at the 5′ end by a methylated guanosine triphosphate, m7Gppp, in contrast to RNA transcribed by RNA polymerase III, which is capped with a methylated gamma phosphate (mpppG). The cap generally marks the mRNA for subsequent processing and nucleocytoplasmic transport, protects the transcript from degradation, and promotes efficient initiation of protein synthesis (Varani, 1997), though some pol II transcripts have m2,2,7GpppG (tri methylated cap) and are not translated.

In vitro transcribed capped RNA mimics most eukaryotic mRNAs found in vivo, because it has a 7-methyl guanosine cap structure at the 5′ end. Capping reactions are performed concomitantly with transcription reactions. Capping reaction protocols are well known to those of ordinary skill in the art. Examples can be found in Sambrooke et al., 2001 and 1989, as well as in U.S. Pat. Nos. 6,511,832 and 6,111,095, all of which are specifically incorporated by reference herein.

In addition to the decreased yields obtained by introducing cap analog into a transcription reaction, 30-50% of the “capped” RNA synthesized by in vitro transcription with cap analog contains the cap in the reverse orientation (Pasquinelli, 1995). Reverse-capped RNA is exported two to three times more slowly from nucleus to cytoplasm than properly capped RNA. Other investigators noted that the presence of reverse caps reduced translational efficiency (Stepinski, 2001). These same investigators designed two novel cap analogs that are incapable of being incorporated in the reverse orientation, anti-reverse cap analog (ARCA, m7,3′OmeGpppG, m7,3′dGpppG). Thus, in some embodiments, a cap analog that dictates proper orientation is employed.

Other candidates for a cap analog include m7GpppA, m7GpppC, dimethylated cap analog (m2,7GpppG), trimethylated cap analog (m2,2,7GpppG), dimethylated symmetrical cap analogs (m7Gpppm7G), 2′ modified ARCA (m7,2′OmeGpppG, m7,2′dGpppG, Jemielty et al., 2003) and ARCA tetraphosphate derivatives (Jemielty et al., 2003).

Kits are also available for preparing capped RNA transcripts. Such transcripts can be synthesized with Ambion's mMESSAGE mMACHINE® Kit. mMESSAGE mMACHINE® reactions include cap analog [m7G(5′)ppp(5′)G] in a high-yield transcription reaction. The cap analog is incorporated only as the first or 5′ terminal G of the transcript because its structure precludes its incorporation at any other position in the RNA molecule. mMESSAGE mMACHINE® Kits have a simplified reaction format in which all four ribonucleotides and cap analog are mixed in a single solution. The cap analog:GTP ratio of this solution is 4:1, which the instructions for this kit indicate is optimal for maximizing both RNA yield and the proportion of capped transcripts. However, the present invention improves upon this technology to produce even higher concentrations of capped RNA.

It may be desirable to incorporate a non-cap, non-extending nucleotide at the 5′ end of a transcript. Thus, it is contemplated that 5′-hydroxy, mono- and di-phosphate nucleotides can be employed instead of a cap structure in methods of the invention. Examples include guanosine 5′-monophosphate disodium salt hydrate (Sigma-Aldrich cat. #51090) and guanosine 5′-diphosphate disodium salt (Sigma-Aldrich cat. #51060). Other such nucleotides are well known to those of skill in the art.

The efficient capping method of the invention is compatible for use with other commercially available kits, such as those used for generating RNA transcripts. The invention can be used with components of such kits to produce a high yield of capped RNA. Any of the compositions described herein may be comprised in a kit or used with kits already commercially available. In a non-limiting example, reagents for producing RNA transcripts and capping those transcripts with a cap structure are provided by Ambion's mMessage mMACHINE® kits. However, because methods of the invention contemplate large-scale reactions (on the order of milligrams to grams of reaction product), it is contemplated that the reagents found in commercially available kits may be employed, but in much higher amounts. The mMESSAGEmMACHINE® kit includes: RNA polymerase (SP6, T7, or T3) in buffered 50% glycerol with SUPERase•In™; 10× Reaction Buffer containing at least salts, buffer, dithiothreitol; 2× NTP/CAP in a neutralized solution containing either 1) ATP (10 mM), CTP (10 mM), UTP (10 mM), GTP (2 mM) and cap analog (8 mM) or 2) ATP (15 mM), CTP (15 mM), UTP (15 mM), GTP (3 mM) and cap analog (12 mM); GTP (either 20 mM or 30 mM); DNase 1 (2U/μl); control template; Ammonium Acetate Stop Solution (5 M ammonium acetate, 100 mM EDTA); Lithium Chloride Precipitation Solution (7.5 M lithium chloride, 50 mM EDTA); nuclease-free water; and Gel Loading Buffer for denaturing gels (95% formamide, 0.025% xylene cyanol, 0.025 bromophenol blue, 18 mM EDTA, 0.025% SDS).

In specific embodiments of the invention, GTP is the component added to a transcription and capping reaction by a fed batch process. It is contemplated that GTP analogs may also be used. GTP analogs that are not cap analogs are well known to those of skill in the art, and may include, but are not limited to, 8-deaza GTP and α-thio GTP.

For the large-scale reactions included in the invention, it is contemplated that the concentration of reagents provided would differ than those previously available. In some embodiments, reagents may be provided, in a kit or not, as follows: 2× NTP/Cap structure mixture (12-15 mM of ATP, UTP, and CTP; 0.5-1 mM GTP; and 6-12 mM of cap analog); additional tube of concentrated GTP (100-200 mM) for addition to the reaction, for example, 0.25-0.5 mM every 5 to 10 minutes.

II. Fed-Batch Process and Other Supplementation Processes

The present invention involve implementing, in some embodiments, a “fed-batch” process to increase the efficiency of a reaction involving competing components. All reaction components are initially combined, but then additional amounts of one or more of the reagents, particularly at least one of the competing components, were added over time, to try to maintain constant reaction conditions

The fed-batch process was originally used in the context of cell culture. Fed-batch culture was different from simple-batch culture, in which all components for cell culturing (including the cells and all culture nutrients) are supplied to the culturing vessel at the start of the culturing process. A fed-batch culture is also different from perfusion culturing insofar as the supernatant is not removed from the culturing vessel during the process (in perfusion culturing, the cells are restrained in the culture by, e.g., filtration, encapsulation, anchoring to microcarriers, etc., and the culture medium is continuously or intermittently introduced and removed from the culturing vessel). See U.S. Pat. No. 6,610,516, which is hereby incorporated by reference herein. Application of the fed-batch process to an enzymatic reaction is contemplated as part of the invention.

The fed-batch process can be implemented manually, semi-automatically, or automatically so long as the device can accurately deliver volumes on the order of microliters. It can involve a device that periodically provides the additional amounts of components to the reaction. The invention is not limited by the particular device used to implement methods of the invention. Such devices can be readily obtained or manufactured. For example, a liquid handler robot could be used to deliver reagents to reactions in 96 well plates.

In other embodiments, the fed-batch process is implemented indirectly, such as by supplementing the reaction with components indirectly. These embodiments can involve non-reacting physical structures that ultimately control the amount of a component that is available for the reaction. Such physical structures include beads, membranes, and other barriers.

Alternatively, the amount of a reaction component may be dictated by the amount of that component available for the reaction as controlled by one or more agents. The agents could be ones that control the amount of one or more phage polymerase substrates produced from a precursor, for example, enzymes that generate nucleoside triphosphate from a nucleoside monophosphate, as described in published U.S. Patent Application 20030113778, which is hereby incorporated by reference.

The invention concerns the supplementation of a competing product to a reaction, and thus, is not limited by the way in which the fed-batch process is implemented.

A continuous flow of one or more reaction components may be employed to supplement the transcription and capping reaction. This may involve fully automated, semi-automated, or manual devices to implement the continuous flow. Typically, the automated devices can be programmed to supplement the reaction at a particular rate. Such devices are well known to those of skill in the art, such as the Microlab 500A, 500B, 500C, 500BP (from Hamilton) and the SP100i, 200i, 250i, 310i (from WPI).

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Standard mMmM In Vitro Transcription Reaction

A standard mMESSAGE mMACHINE® T7 Kit reaction contains 50 ng/μl plasmid template; 4 U/μl T7 RNA polymerase; 0.005 U/μl IPP; 0.03 U/μl RNase Inhibitor; 0.01 U/μl SUPERase•In; 0.1% Chaps; 40 mM Tris, pH 8.0; 20-30 mM MgCl₂; 2 mM spermidine; 10 mM DTT; 7.5 mM ATP; 7.5 mM CTP; 7.5 mM UTP; 1.5 mM GTP; and, 6 mM m7GpppG cap analog. Components are assembled at room temperature in a final volume of 20 μl and the reaction is incubated at 37° C. up to two hours. Under these conditions, transcription reactions with pTRI-Xef (˜1.8 kb RNA), pAmbluc (˜1.8 kb RNA) or p4kb (˜4 kb RNA) templates produce ˜30 μg of transcript (˜1.5 mg/ml) in 30 minutes. A time course study with the p4kb template is presented in FIG. 1. Analysis of the RNA produced on a RNA LabChip with the Agilent 2100 bioanalyzer showed no RNA degradation over time.

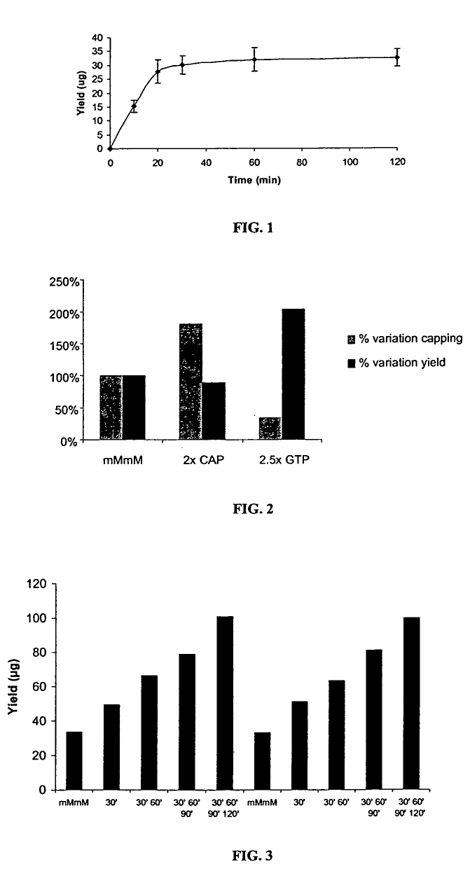

The cap:GTP ratio in a standard mMmM reaction is 4:1. Analysis of the capping efficiency confirmed that less than approximately 80% of the produced RNA is capped. To improve capping efficiency, the reaction was performed in the presence of more cap analog. For example with 12 mM m7GpppG (8:1 ratio) the capping efficiency is ˜180% that of a standard mMmM reaction (2× CAP, FIG. 2). However this approach is not cost effective as m7GpppG is one of the most expensive reagent and only a fraction is used in a standard mMmM reaction (less than 1% for transcript larger than 100 nt).

To improve transcription yield, the GTP concentration in a standard mMmM reaction was increased. For example with 3.75 mM GTP, the yield is ˜65 μg, corresponding to a 215% increase over standard mMmM reaction (2.5× GTP, FIG. 2). However the cap:GTP ratio in this reaction is 1.6:1 resulting in a very poor capping efficiency.

### Example 2

### Fed-Batch In Vitro Transcription Reaction—Manual Feed

The time course study presented in FIG. 1 shows that a standard mMmM reaction is essentially completed after 30 min incubation at 37° C. At this time point, most of the GTP has been incorporated into the transcript. In contrast, only a small fraction of the cap analog has been used as 1) there is a 4-fold excess of cap over GTP in the reaction, 2) only 1 molecule of cap is incorporated per molecule of RNA synthesized, and 3) only ˜80% of the transcripts are capped. The amount of GTP used and the percentage of cap used in the reaction can be easily estimated from the yield and the size of the transcript. The average molecular weight of a given RNA molecule is equivalent to its total number of residues. If this value is multiplied by 320 g/mol (the average molecular weight for all 4 residues), then:
[GTP] used in mM=(3.12×yield×G)/nt
% cap used=(250×yield)/(nt×[cap])
with yield in μg/μl or mg/ml
G=number of G residues per transcript
nt=total number of residues per transcript
[cap]=initial cap analog concentration in mM

Thus 1-1.25 mM GTP is used after 30 min in a standard mMmM reaction, corresponding to 25-32 μg transcript synthesized. In contrast, less than 0.02% of the cap analog is consumed in the same time with the p4kb template; less than 0.04% with the shorter pTRI-Xef or pAmbluc templates. As the cap concentration is still ˜6 MM after 30 min incubation, addition of 20 nmol of GTP to the reaction would increase the GTP concentration by 1 mM without significantly affecting the cap:GTP ratio.

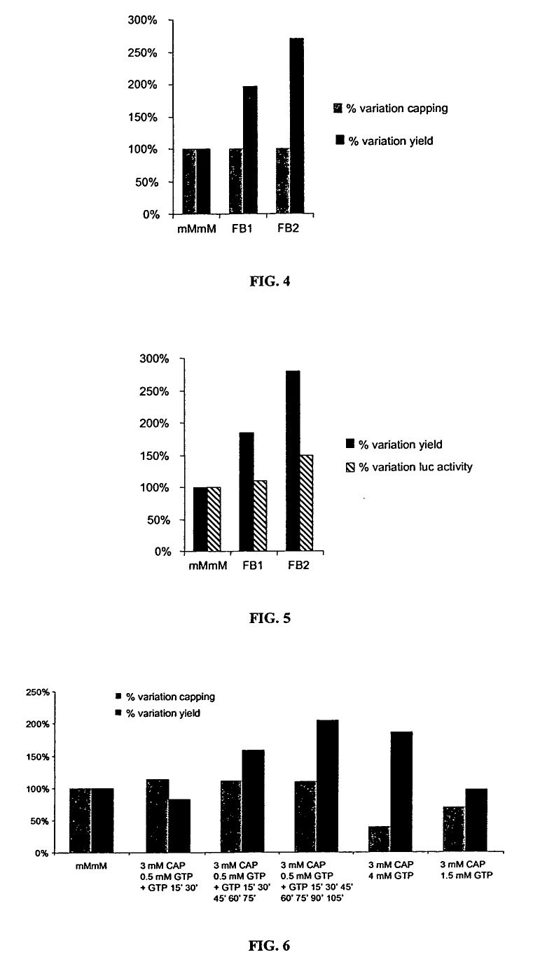

Using this approach, the yield of a standard mMmM can be significantly increased without affecting the capping efficiency (30′, FIG. 3). The same strategy can be repeated several times. For example, after addition of 4 mM GTP by 1 mM increment every 30 min, a standard mMmM reaction yields ˜5 mg/ml capped RNA (30′ 60′ 90′ 120′, FIG. 3). Similar results were obtained by adding a smaller amount of GTP earlier in the reaction, e.g., 0.5 mM GTP every 15 min (see for example FBI in FIGS. 4 and 5). Analysis of the produced RNA with the RNA LabChip and the capping assay confirmed that the quality and the capping efficiency were not affected by the fed-batch strategy.

### Example 3

### Fed-Batch In Vitro Transcription Reaction—Other Cap Analogs

Any non-extending, mono- or di-nucleotide (i.e., that cannot be incorporated as a 3′ nucleotide in a transcription reaction) can be incorporated as the first nucleotide of a transcript by phage RNA polymerases, and are compatible with the fed-batch strategy. This includes 5′ hydroxyl, monophospate or biotinylated nucleotides, trimethylated cap analog (m2,2,7GpppG), unmethylated cap variant (GpppG), tetraphosphate cap variant (m7GppppG) or other cap variants (e.g. m7GpppA, m7GpppC). Of particular interest are the anti-reverse cap analogs (ARCAs). With the standard cap analog m7GpppG, because of the presence of a 3′-OH on both the m7Guanosine and Guanosine moieties, 30-50% of the initiating dinucleotide is incorporated in a reverse, non-functional orientation (Pasqinelli et al., 1995). ARCA molecules such as m7dGpppG, m7,3′-OMeGpppG, m7,3′-OMeGppppG or m7,2′-OMeGppppG (Stepinski et al., 2001; Jemielity et al., 2003) are modified at the 3′-O or 2′-O position of m7Guanosine and cannot be incorporated in the reverse orientation. Some of these modifications do not affect cap-dependent translation and strongly enhance translation efficiency in vivo. For example, the luciferase activity resulting from luciferase mRNA capped with m7,3′-OmeGpppG and transfected in HeLa cells was 2-4 fold higher than with mRNA capped with standard cap analog. Another “ARCA-like” strategy is to use a symmetrical, dimethylated cap analog (m7Gpppm7G). This analog was efficiently incorporated during in vitro transcription and increased luciferase activity by 1.5-fold in vivo.

An example of fed-batch reaction with the ARCA m7,3′-OmeGpppG and the p4kb template is provided in FIG. 4. In this experiment, two different feeding methods were tested. In FB1, 10 nmol of GTP was added after 15, 30, 45 and 60 min incubation at 37° C. In FB2, 20 nmol GTP was added at 30, 60, 90 and 120 min. With both methods the expected increase in RNA yield was observed without affecting the capping efficiency.

### Example 4

### Fed-Batch In Vitro Transcription Reaction—Biological Activity

Capped mRNA encoding specific genes can be transfected into eukaryotic cells or microinjected into cells or embryos. Such approaches are used to study the effect of the corresponding translated product, to express reporter proteins (e.g, luciferase or GFP) or in therapeutic strategies (e.g., production of non-infectious, vaccine virus or immunotherapy with dendritic cells). Thus, it is critical that mRNA produced by in vitro transcription are not only efficiently capped, but also efficiently translated in vivo.

To evaluate the biological activity of capped mRNA synthesized with the fed-batch strategy, mRNAs encoding the firefly luciferase gene were prepared using a standard mMmM reaction or the two fed-batch reactions described above (FB1 and FB2). Similar to the p4kb template (FIG. 4), transcription yields with the pAmbluc template increased by ˜200 and 375% with the FB1 and FB2 methods, respectively (FIG. 5). After transfection in HeLa cells, the luciferase activity from mRNA prepared by fed-batch reactions was equivalent or higher than from mRNA prepared with the mMmM protocol (FIG. 5). This result confirms that cap analogs incorporated in transcripts by fed-batch in vitro transcription are functional.

### Example 5

### Fed-Batch In Vitro Transcription Reaction—Changing Cap Concentration

With the FB1 method described above, 0.5 mM GTP was added every 15 min, keeping the GTP concentration at ˜1.5 mM, and therefore, the cap:GTP ratio at ˜4:1. The same strategy can be used starting with less GTP, therefore keeping the GTP lower and the cap:GTP ratio higher. For example, a fed-batch reaction with the pAmbluc template, started with 6 mM cap analog and 0.5 mM GTP (12:1 ratio), with four successive additions of 0.5 mM GTP every 15 min, yielded more full length capped RNA than a standard mMmM reaction. A further embodiment is to start the reaction with less cap analog and to feed GTP at low level to keep the cap:GTP ratio equivalent or higher than in a standard mMmM reaction (4:1). This is especially important as cap analog is the most expensive reagent in a transcription reaction and only a very small fraction is used (less than 1% for transcript larger than 100 nt).

The results of such a strategy is presented in FIG. 6 where fed-batch reactions were started with 0.5 mM GTP, 3 mM cap analog and 1, 2.5 or 3.5 mM total GTP was added by 0.5 mM increments every 15 min. With the addition of 3.5 mM GTP, the final yield was ˜210% compared to the control standard mMmM reaction. As the cap:GTP ratio was higher than in a standard mMmM reaction (˜6:1 vs 4:1), the expected increase in capping efficiency was observed. In contrast, batch reactions started with 3 mM cap analog and 1.5 or 4 mM GTP (2:1 or 3:4 ratio) yielded poorly capped transcripts. In conclusion the fed-batch strategy not only increased the overall yield of the transcription reaction but also improved the capping efficiency while using less cap analog.

### Example 6

### Fed-Batch In Vitro Transcription Reaction—Automatically Fed

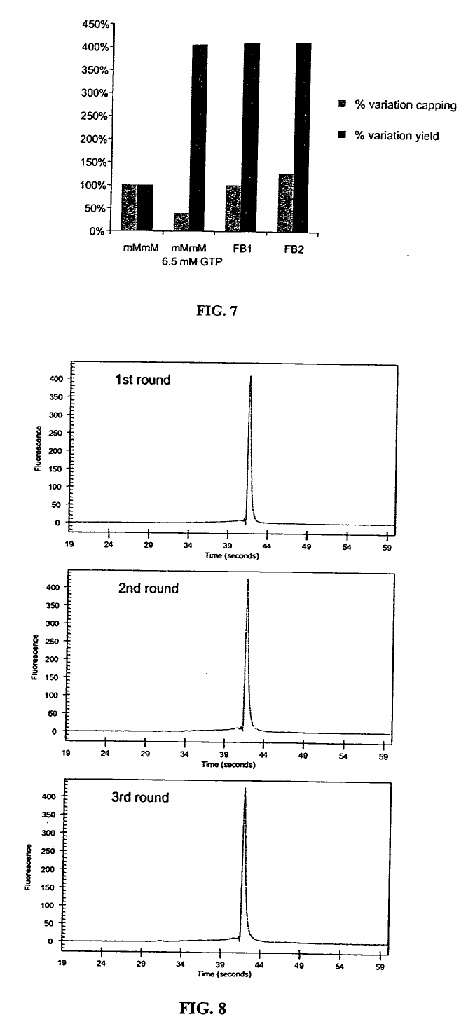

The above results show that the cap:GTP ratio can be artificially kept high in the fed-batch strategy by starting the reaction at very low GTP concentration and adding small amount of GTP. To further improve the procedure, an automatic or semi-automatic dispensing device can be used to add small volume of GTP at regular intervals over a longer period of time. In this example, a syringe pump controlled by a computer was used to implement the fed-batch process with 100 μl in vitro transcription reactions (FIG. 7). Reactions were initiated with 3 mM m7GpppG and 0.5 (FB1) or 0.25 (FB2) mM GTP. 0.5 or 0.25 mM GTP was then added to the respective reactions every 10 or 5 min for two hours, resulting in a total amount of GTP equivalent to 6.5 and 6.25 mM GTP. FB1 and FB2 RNA yields were increased by more than 400% over a standard mMmM reaction. As expected the capping efficiency was better with the FB2 method (higher cap:GTP ratio) while RNA synthesized by a batch method initiated with 6.5 mM GTP were poorly capped (FIG. 7).

### Example 7

### Fed-Batch In Vitro Transcription Reaction—Other Phage Polymerases

Other phage polymerases are compatible with the fed-batch strategy. As an example, 100 μl reactions were performed using the FB2 method described in FIG. 7, recombinant T3 RNA polymerase (Ambion) and the linearized pTRi-Xef template (the pTRI vector carries the T3, T7 and SP6 promoters in the same orientation). The reactions consistently yielded 500-600 μg of RNA, similar to fed-batch T7 reactions or to control batch T3 reactions in the presence of 6.5 mM GTP.

### Example 8

### Large-Scale, Bovine-Free, Fed-Batch In Vitro Transcription Reaction

In the past 5 years, several clinical trials have been initiated to evaluate the safety and efficacy of a variety of innovative RNA-base therapies. These strategies will require large quantities of capped RNA manufactured under the US Current Good Manufacturing Practice (21 CFR 210, 211, 600, Part 11) and in accordance to the FDA Quality System Requirement (21 CFR, Part 820). The fed-batch method was tested using ampicillin- and bovine-free components. The T7 and IPP enzymes were expressed from vectors that encode the kanamycin resistance gene. A 10 ml reaction was set up with 500 μg of linearized p4kb plasmid template, 40,000 units recombinant T7 RNA polymerase, 50 units recombinant IPP6.5 mM ATP, 6.5 mM CTP, 6.5 mM UTP, 0.25 mM GTP and 3 mM m7GpppG cap analog. After addition of 6 mM GTP by 0.25 mM increments every 5 minutes for 2 hours, the reaction yield about 60 mg of full length capped RNA.

### Example 9

### PCR Template and Immobilized Template

PCR products are efficient templates for fed-batch in vitro transcription reactions. A promoter can be added to the PCR product by including the promoter sequence at the 5′ end of either the forward or reverse PCR primer. These bases become a double-stranded promoter sequence during the PCR reaction. The use of PCR products in transcription reactions reduces the somewhat long and tedious cloning, plasmid purification and plasmid linearization steps. A further improvement is to use modified PCR products or plasmids that can be subsequently immobilized on a solid support. Such templates can be reused several times and considerably reduce the amount of residual DNA contamination in the transcription reaction.

In this example, a 1.7 kb luciferase PCR fragment was amplified in the presence of a 5′ biotinylated forward primer containing the T7 promoter sequence, and then bound on streptavidin beads. The immobilized template was used in 3 successive fed-batch reactions, each initiated with 3 mM m7GpppG and 0.25 mM GTP with addition every 5 min of 0.25 mM GTP for two hours. Each reaction produced about 500 μg of transcript with no reduction of RNA yield or RNA quality (FIG. 8). Overall 1.2 mg of RNA was synthesized per μg of DNA template.

### Prophetic Example 10

### Feeding Additional Components

As nucleotides are consumed during the transcription reaction, the reaction conditions can be modified:
(RNA)ⁿ⁻+MgNTP²⁻→(RNA)⁽ⁿ⁺¹⁾⁻+MgP₂O₇²⁻+H⁺
Thus, one of the major changes is a drop in pH resulting from the production of one proton for each nucleotide incorporated in the transcript. Another byproduct of the transcription reaction is inorganic pyrophosphate ions (P₂O₇⁴⁻). In absence of inorganic pyrophosphatase enzyme (IPP), pyrophosphate ions are complexed with magnesium, forming a white Mg₂P₂O₇ precipitate and resulting in a progressive reduction of free magnesium concentration. To further improve the fed-batch reaction and maintain optimal transcription conditions over time, other components can be fed in addition to the otherwise limiting, competing component (GTP). These components include, but are not limited to, magnesium salt to maintain the concentration of free magnesium, OH⁻ ions to increase the pH or other nucleotides that may become limiting over time.

### Prophetic Example 11

### Other Feeding Methods

In addition to automatic or semi-automatic dispensing devices that inject the limiting nucleotide(s) in the fed-batch reaction, other methods can be used for continuously or semi-continuously adding the desired component(s). Another method may be a bead-feed. All of the components of the transcription reaction will be combined in the presence of a bead or some other device that slowly and continuously delivers nucleotide(s) and other components to the reaction. The components may be formulated in non-reactive matrix (such as cellulose) that slowly dissolves during the reaction. Alternatively, the components may be encapsulated in a hollow bead with a small hole. The components would slowly leak out into the reaction.

Instead of adding the desired component(s) directly to the fed-batch reaction, it could be provided as a substrate for an enzyme or an enzymatic pathway that would then produce the limiting reaction component, such as nucleotide. The substrate itself could not be incorporated into the reaction product until it had been converted to the limiting reaction component. For example, instead of adding GTP to the reaction, GMP together with nucleoside monophospate and diphosphate kinases are added to the reaction. The GMP is not incorporated into the RNA. However, it can be converted to GTP that is then incorporated into the RNA. The rate of GMP to GTP conversion could be controlled by the concentration of GMP and kinases. As the GTP is utilized by transcription, more GMP will be converted for incorporation into the reaction product

All of the compositions and/or methods and/or apparatus disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and/or apparatus and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references are specifically incorporated herein by reference.
U.S. Pat. No. 5,256,555
U.S. Pat. No. 5,256,555
U.S. Pat. No. 6,111,095
U.S. Pat. No. 6,511,832
U.S. Pat. No. 6,610,516
U.S. patent application Ser. No. 20030113778
Ausubel et al., In: Current Protocols in Molecular Biology, John, Wiley & Sons, Inc, New York, 1996.
Burkhard et al., Vaccine, 21(3-4):258-268, 2002.
Coleman et al., Nucleic Acids Res., 32(1):e14, 2004.
Davis et al, IUBMB Life, 53(4-5):209-211 2002.
Eiben et al., Cancer Res., 62(20):5792-5799, 2002.
Geisbert et al., Virology, 293(1):15-9, 2002.
Gurevich et al., Anal Biochem., 195(2):207-213 1991.
Heiser et al., J. Clin. Invest., 109:409-417, 2002.
Hevey et al., Virology, 251(1):28-37, 1998.
Jemielty et al., RNA, 9(9):1108-1122, 2003.
Kern et al., Biotechnol. Prog., 15:174-184, 1999.
Kern et al., Biotechnol. Prog., 13:747-756, 1997.
Krieg and Melton, Methods Enzymol., 155:397-415, 1987.
Lee et al., Infect. Immun., 69:5709-5715, 2001.
Lee et al., Infect. Immun., 71:1491-1496, 2003.
Morse et al., Int. J. Gastrointest. Cancer, 32:1-6, 2002.
Nielsen et al., Nucleic Acids Res. 14(14):5936, 1986.
Pasquinelli et al., RNA, 1:957-967, 1995.
Pushko et al., J. Virol., 75:11677-11685, 2001.
Pushko et al., Virology, 239:389-401, 1997.
Sambrook et al., In: Molecular cloning, Cold Spring Harbor Laboratory Press, Cold Spring Harbor, N.Y., 2001.
Sambrook et al., In: Molecular cloning, Cold Spring Harbor Laboratory Press, Cold Spring Harbor, N.Y., 1989.
Sampson and Uhlenbeck, Proc. Natl. Acad. Sci. USA, 85(4):1033-1037, 1988.
Stepinski et al., RNA, 7:1486-1495, 2001.
Sullenger and Gilboa, Nature, 418:252-257, 2002.
Wyatt et al., Biotechniques, 11(6):764-769, 1991.
Yisraeli et al., Methods Enzymol., 180:42-50, 1989.

1. A method for producing capped RNA comprising:
a) incubating components for a transcription and capping reaction under conditions to promote transcription and capping, wherein the components include a cap analog, a nucleotide that competes with the cap analog, and non-competing nucleotides; and,
b) supplementing the reaction with the competing nucleotide to maintain the concentration of the competing nucleotide in the reaction at a ratio between about 1:1 and about 1:50 relative to the concentration of the cap analog in the reaction.

2. The method of claim 1, wherein the reaction is supplemented to maintain the concentration of the competing nucleotide in the reaction at a ratio between about 1:4 and about 1:25 relative to the concentration of the cap analog in the reaction.

3. The method of claim 1, wherein the reaction is supplemented intermittently by a fed batch process.

4. The method of claim 1, wherein the competing nucleotide is GTP or a GTP analog.

5. The method of claim 1, wherein the concentration of the cap analog in the reaction is between about 1 mM and about 10 mM.

6. The method of claim 5, wherein the concentration of the cap analog in the reaction is between about 2 mM and about 6 mM.

7. The method of claim 3, wherein the reaction is supplemented at least two times by the fed-batch process.

8. The method of claim 1, wherein the reaction is supplemented with the competing nucleotide to maintain the concentration of the competing nucleotide in the reaction between about 0.1 mM and about 2.0 mM.

9. The method of claim 3, wherein each supplementation by the fed-batch process adds between about 0.1 mM and about 2.0 mM of the competing nucleotide to the reaction.

10. The method of claim 9, wherein each supplementation by the fed-batch process adds between about 0.2 mM and about 1 mM of the competing nucleotide to the reaction.

11. The method of claim 1, wherein the reaction is supplemented with other components of the reaction but not all components of the reaction.

12. The method of claim 1, wherein the reaction yields between about 1 mg/ml and about 10 mg/ml of capped transcript.

13. The method of claim 12, wherein the reaction yields between about 4 and about 7 mg/ml of capped transcript.

14. The method of claim 3, wherein the supplementation is periodic.

15. The method of claim 1, wherein the supplementation is continuous during most of the reaction.

16. The method of claim 15, wherein the supplementation of the competing nucleotide is at a rate of about 10 μM per minute to about 200 μM per minute.

17. A method for producing capped RNA comprising introducing to a transcription and capping reaction GTP or a GTP analog by a fed-batch process.

18. The method of claim 17, wherein other reaction components, except a cap analog, are introduced to the reaction by the fed-batch process.

19. The method of claim 17, wherein the concentration of GTP introduced into the reaction depends on the initial concentration of a cap analog in the reaction.

20. The method of claim 19, wherein the concentration of GTP introduced into the reaction is determined based on a ratio of the concentration of GTP to the concentration of the cap analog, wherein the ratio is between about 1:1 and about 1:50.

21. (canceled)

22. The method of claim 17, wherein the amount of GTP or a GTP analog introduced in the reaction by the fed-batch process increases the concentration of GTP or GTP analog in the reaction by less than about 4 mM after each introduction.

23-24. (canceled)

25. The method of claim 17, wherein the initial concentration of a cap analog in the reaction is between about 1 mM and about 10 mM.

26-30. (canceled)

31. The method of claim 17, wherein the cap analog is selected from the group consisting of m7GpppG; m7GpppA; m7GpppC; GpppG; m2,7GpppG; m2,2,7GpppG; m7Gpppm7G; ARCA; and, m7,2′OmeGpppG, m72′dGpppG, m7,3′OmeGpppG, m7,3′dGpppG and their tetraphosphate derivatives.

32-33. (canceled)

34. The method of claim 17, wherein the initial reaction volume is at least about 100 μl.

35-37. (canceled)

38. The method of claim 17, wherein one or more of the following components is also introduced by the fed-batch process: polymerase, pyrophosphatase, a magnesium salt, or a ribonuclease inhibitor.

39. The method of claim 17, wherein GTP or a GTP analog are introduced into the reaction by a fed-batch process so as to maintain the concentration of GTP or a GTP analog in the reaction less than about 1 mM.

40. The method of claim 17, wherein introduction of GTP or a GTP analog by the fed-batch process is intermittent or periodic.

41-46. (canceled)

47. The method of claim 17, wherein one of more reaction components are immobilized.

48. The method of claim 47, wherein template is immobilized.

49-60. (canceled)

61. A method for producing transcripts with a nonextending nucleotide at the 5′ end comprising introducing a nucleotide that competes with the nonextending nucleotide by a fed-batch process to a transcription reaction comprising RNA polymerase and the non-extending nucleotide.

62-65. (canceled)
